(19) United States
(12) Patent Application Publication
(10) Pub. No.: US 2003/0165714 A1
(43) Pub. Date: Sep. 4, 2003

(54) Red organic electroluminescent compounds, method for synthesizing the same and electroluminescent devices

(76) Inventors: Jeong-ik Lee, Daejeon (KR); Byung-Jun Jung, Pusan (KR); Hong-ku Shim, Daejeon (KR); Hye-Yong Chu, Daejeon (KR); Lee-mi Do, Daejeon (KR); Tae-hyoung Zyung, Daejeon (KR)

Correspondence Address: BLAKELY SOKOLOFF TAYLOR & ZAFMAN, 12400 WILSHIRE BOULEVARD, SEVENTH FLOOR, LOS ANGELES, CA, 90025, US

(21) Appl. No.: 10/302,040

(22) Filed: Nov. 21, 2002

(30) Foreign Application Priority Data
Nov. 22, 2001 (KR) 2001-73004

(51) Int. Cl. H05B 33/14; C09K 11/06; C 07D 3 9/34

(52) U.S. Cl. 428/690; 428/917; 313/504; 252/301.16; 549/426

(57) ABSTRACT

Red organic electroluminescent compounds containing bis-condensed DCM derivatives, a method for synthesizing the same and an organic electroluminescent device using the same. The red organic electroluminescent compound having the formula:

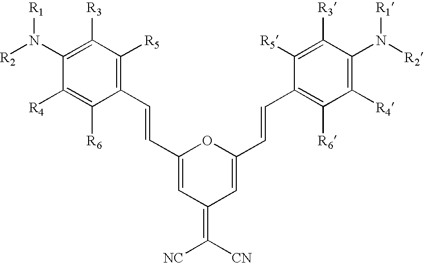

wherein R₁, R₁′, R₂ and R₂′ are independently hydrogen atom, or C_{1—}C₃₀ alkyl, aryl or hetero ring; R₃, R₃′, R₄ and R₄′ are independently hydrogen atom, C_{1—}C₁₀ alkyl or alkoxy; one or more pairs selected from the group consisting of R₁ and R₃, R₁′ and R₃′, R₂ and R₄, and R₂′ and R₄′ can be connected in forms of —R₁-R₃—, —R₁′-R₃′—, —R₂-R₄—, and —R₂′-R₄′—; R₅, R₅′, R₆ and R₆′ are independently hydrogen atom, or C_{1—}C₃₀ alkyl, alkoxy or aryl; at least one of R₃, R₃′, R₄, R₄′, R₅, R₅′, R₆ and R₆′ is not hydrogen atom.

This application claims the priority of Korean Patent Application No. 2001-73004, filed Nov. 22, 2001, in the Korean Intellectual Property Office, the disclosure of which is incorporated herein in its entirety by reference.

### BACKGROUND OF THE INVENTION

1. Field of the Invention

The present invention relates to red organic electroluminescent compounds, a method for synthesizing the same and electroluminescent devices. More particularly, the present invention relates to red organic electroluminescent compounds having a 4-(dicyanomethylene)-2-methyl-6-(p-dimethylaminostyryl)-4H-pyran (DCM) derivative, a method for synthesizing the same, and electroluminescent devices using the same.

2. Description of the Related Art

Since organic electroluminescent devices having a thin film made of an organic material was disclosed by Ching W. Tang et al. in U.S. Pat. No. 4,539,507 and Appl. Phys. Lett., vol. 51, page 913 (1987) in the late 1980, techniques of fabricating devices by doping fluorescent materials into an emitting layer have been widely used for achieving emissions of primary colors of light, that is, red, green and blue, necessary for color display (Appl. Phys. Lett., Vol. 65, page 3610 (1989)).

In particular, red emissions with low luminescence efficiency and insufficient color purity have many difficulties in applications. Typical materials originally used to provide the red emissions were DCM and julolidyl derivatives thereof (DCJ) (Appl. Phys. Lett., Vol. 65, page 3610 (1989)). However, among the three primary colors, red with the lowest luminescence efficiency has become considered as the most serious obstacle to attainment of full-color display panels.

In order to solve the problem, developments in luminescent materials with higher luminescence efficiency have been disclosed in U.S. Pat. No. 5,908,581, and Macromol. Symp., Vol. 125, page 49 (1997). However, a mono-condensed DCJ derivative could not provide pure red with 1931 CIE color coordinates of x=0.67 and y=0.33, requested by the National Television System Committee (NTSC). Also, it has been known that red luminescent devices experienced sharply decreasing luminescence efficiency with increasing dopant concentration (Chem. Phys. Lett. Vol. 287, page 455 (1998) and Thin Solid Films, Vol. 363, page 327 (2000)). Accordingly, in order to realize a pure red electroluminescent device, it is necessary to prepare a fluorescent material capable of providing red emission at a low doping concentration and having high luminescent efficiency.

It has been hitherto known that bis-condensed derivatives of DCM had very low luminescence efficiency (Optics Comm., Vol 29, page 331 (1979)), seemingly involving the problem of impossibility to be utilized as red light emitting materials, which is, however, resulted from bis-DCJ. Thus, in order to fully utilize bis-condensed derivatives of DCM as red light emitting materials, it is necessary to develop DCM derivative having sufficiently high luminescence efficiency.

### SUMMARY OF THE INVENTION

It is an aspect of the present invention to provide novel red organic electroluminescent compounds having pure red light emitting properties and good luminescence efficiency.

It is another aspect of the present invention to provide a method for synthesizing novel red organic electroluminescent compounds having pure red light emitting properties and good luminescence efficiency.

It is still another aspect of the present invention to provide organic electroluminescent devices which can be industrially advantageously used by employing an emitting layer comprising red organic electroluminescent compounds having pure red light emitting properties and good luminescence efficiency.

In an aspect, the present invention provides a red organic electroluminescent compound having the formula 1:

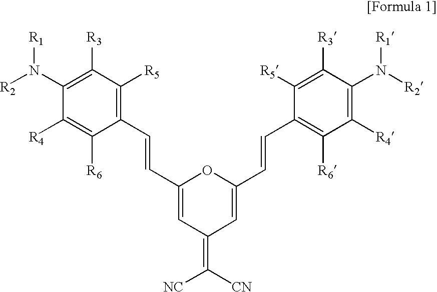

wherein R₁, R₁′, R₂ and R₂′ are independently hydrogen atom, or C_{1—}C₃₀ alkyl, aryl or hetero ring; R₃, R₃′, R₄ and R₄′ are independently hydrogen atom, C_{1—}C₁₀ alkyl or alkoxy; one or more pairs selected from the group consisting of R₁ and R₃, R₁ ′ and R₃′, R₂ and R₄, and R₂′ and R₄′ can be connected in forms of —R₁-R₃—, —R₁′-R₃′—, —R₂-R₄—, and —R₂′-R₄′—; R₅, R₅′, R₆ and R₆′ are independently hydrogen atom, or C_{1—}C₃₀ alkyl, alkoxy or aryl; at least one of R₃, R₃′, R₄, R₄′, R₅, R₅′, R₆ and R₆′ is not hydrogen atom.

In formula 1, the one or more pairs selected from the group consisting of R₁ and R₃, R₁′ and R₃′, R₂ and R₄, and R₂′ and R₄′ can be connected in forms of —R₁-R₃—, —R₁′-R₃′—, —R₂-R₄—, and —R₂′-R₄′—, thereby forming the structure of —CR₇R₈—(CR₉R₁₀)ₘ—CR₁₁R₁₂—. Here, R₇, R₈, R₉, R₁₀, R₁₁ and R₁₂ are independently hydrogen atom or C_{1—}C₄ alkyl, and m is an integer between 0 and 2.

In formula 1, R₁, R₁′, R₂ and R₂′ are preferably methyl, ethyl, propyl, butyl, pentyl, hexyl, aryl, dialkylfluoryl or heteroaryl.

Also, R₃, R₃′, R₄ and R₄′ are preferably hydrogen atom, methyl, ethyl, propyl, butyl or alkoxy.

Further, R₅, R₅′, R₆ and R₆′ are preferably hydrogen atom, methyl, ethyl, propyl, butyl, methoxy, ethyoxy, butoxy, cyclohexylmethoxy or ethylhexyloxy.

A red organic electroluminescent compound according to a feature of the present invention may have the formula 2:

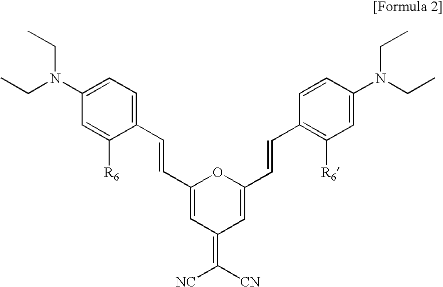

In formula 2, R₆ and R₆′ are preferably methyl, ethyl, methoxy, ethyoxy, propyloxy, butoxy, cyclohexylmethyloxy or ethylhexyloxy.

A red organic electroluminescent compound according to another feature of the present invention may have the formula 3:

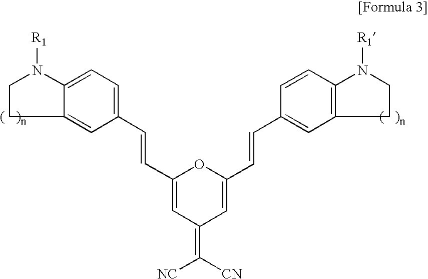

wherein n is an integer between 0 and 3.

In formula 3, R₁ and R₁′ are independently methyl, ethyl, cyclohexyl, hexyl, methylphenyl or dialkylfluoryl, and n is 1 or 2.

In another aspect, the present invention provides a method for synthesizing red electroluminescent compound, including preparing a first compound having the formula 4:

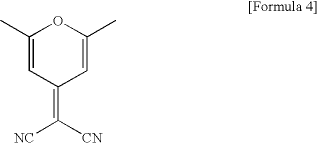

Then, the first compound having the formula 4 is reacted with a second compound having the formula 5:

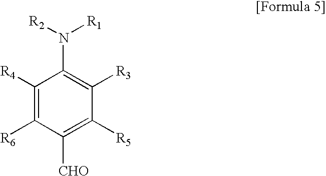

wherein R₁, R₂, R₃, R₄, R₅ and R₆ are as defined above.

In step of reacting the first compound with the second compound, a third compound having the formula 6 can be recovered as a product of the reaction between the first compound and the second compound;

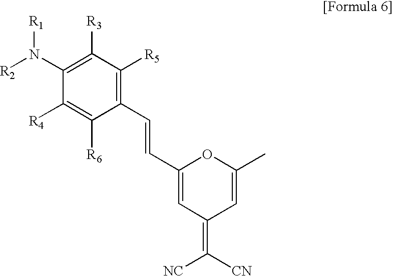

The red organic electroluminescent compound having the formula 1 can be synthesized by reacting the third compound with a fourth compound having the formula 7:

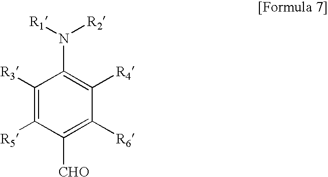

wherein R₁′, R₂′, R₃′, R₄′, R₅′ and R₆′ are as defined above.

In still another aspect, the present invention provides an organic electroluminescent device comprising an anode, a cathode and an emitting layer interposed between the anode and the cathode and having the above-described red organic electroluminescent compound according to the present invention.

The red organic electroluminescent compound can provide pure red light emitting properties and good luminescence efficiency. The organic electroluminescent device having an emitting layer with the red organic electroluminescent compound has good color coordinates, pure red light emitting properties and good luminescence efficiency, thus being industrially advantageously used.

### BRIEF DESCRIPTION OF THE DRAWINGS

The above aspect and advantages of the present invention will become more apparent by describing in detail preferred embodiments thereof with reference to the attached drawings in which:

FIGS. 1A through 1F show synthetic routes illustrating a process of synthesizing red organic electroluminescent compound according to the present invention;

FIG. 2 is a ¹H-NMR spectrum illustrating red organic electroluminescent compounds synthesized in Examples 10 through 18 of the present invention;

FIGS. 3A and 3B are photoluminescent spectra illustrating red organic electroluminescent compounds synthesized in Examples 10 through 18 of the present invention;

FIG. 4 is a cross-sectional view for explaining a method for fabricating an organic electroluminescent device according to Example 19;

FIG. 5 is an electroluminescence (EL) spectrum of the organic electroluminescent devices according to Example 19;

FIG. 6 is a cross-sectional view for explaining a method for fabricating an organic electroluminescent device according to Example 20;

FIG. 7 is an electroluminescence (EL) spectrum of various organic electroluminescent devices according to Example 20;

FIG. 8 illustrates a change in color coordinates as a function of applied currents in the organic electroluminescent devices according to Example 20; and

FIG. 9 is a graphical representation of voltage efficiencies of the organic electroluminescent devices according to Example 20.

### DETAILED DESCRIPTION OF THE INVENTION

In the present invention, in order to synthesize an organic electroluminescent compound capable of realizing red emission with higher purity, a DCM derivative further having an electron donor in an aryl ring having an amine group as an electron donor, is synthesized, thereby obtaining red organic electroluminescent compounds comprising a bis-condensed derivative of DCM capable of providing red emission with good color coordinates.

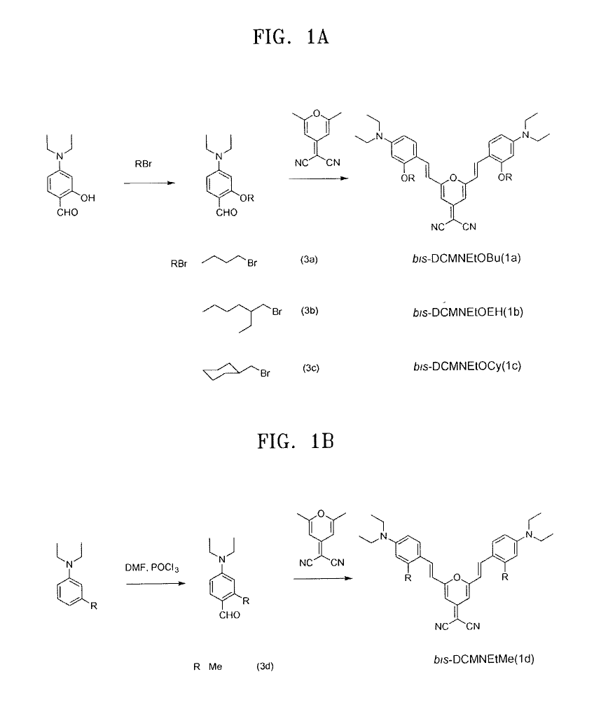

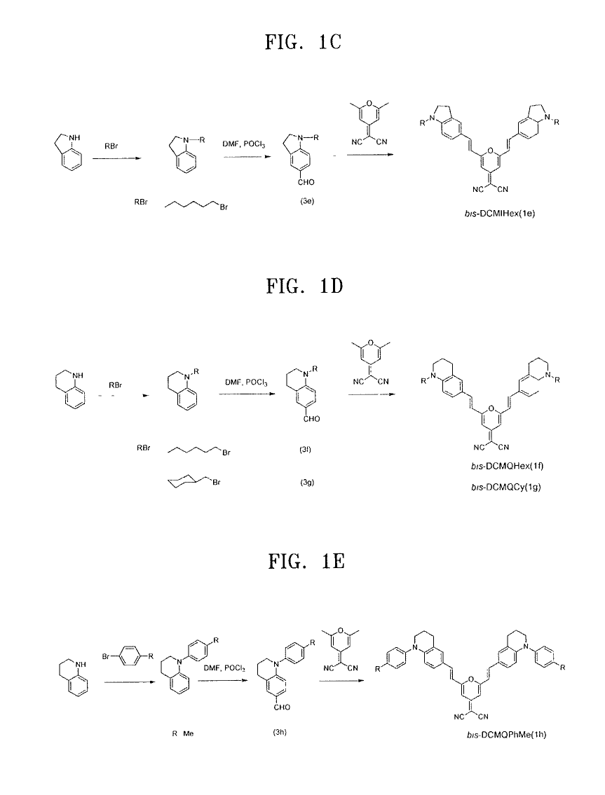

FIGS. 1A through 1F show synthetic routes illustrating a process of synthesizing red organic electroluminescent compound according to the present invention. Referring to FIGS. 1A through 1F, various examples of synthesizing red organic electroluminescent compounds according to the present invention will first be described.

### EXAMPLE 1

### Synthesis of 2,6-dimetyl-4H-pyran-4-ylidine)propanedinitrile (Formula 4)

3.3 g of malononitrile and 6.2 g of 2,6-dimethyl-4-pyrone were boiled with 15 mL acetic anhydride for 8 hours. The resultant reactant was dropped into water, the precipitate was recovered and recrystallized with methanol, to give 6.5 g of 2,6-dimetyl-4H-pyran-4-ylidine)propanedinitrile as a brown solid.

¹H-NMR (CDCl₃): 6.51 (s, 6H). 2.29 (s, 2H)

### EXAMPLE 2

### Synthesis of 4-(diethylamino)-2-butoxybenzaldehyde (3a of FIG. 1A)

3 g of 4-(diethylamino)-2-hydroxybenzaldehyde and 2.5 g of 1-bromobuthane were put into 20 mL DMSO (dimethyl sulfoxide) and 1.5 equivalents of sodium hydroxide was added thereto, followed by heating at 60_ for 8 hours. The resultant reactant was extracted with water and ethylacetate to remove an organic solvent, to give 3.4 g of 4-(diethylamino)-2-butoxybenzaldehyde as a brown liquid in a yield of 89%.

¹H-NMR (CDCl₃): 10.16 (s, 1H), 7.69 (d, 1H), 6.25 (d, 1H), 6.00 (s, 1H), 4.01 (t, 2H), 3.41 (q, 4H) 2.02 (m, 2H), 1.50 (m, 2H), 1.20 (t, 6H), 0.97 (t, 3H)

### EXAMPLE 3

### Synthesis of 4-(diethylamino)-2-(2-ethylhexyloxy)benzaldehyde (3b of FIG. 1A)

5 g of 4-(diethylamino)-2-hydroxybenzaldehyde and 6 g of 2-ethylhexylbromide were put into 30 mL DMSO, and 1.5 equivalents of sodium hydroxide was added thereto, followed by heating at 60_ for 8 hours. The resultant reactant was extracted with water and ethylacetate to remove an organic solvent, to give 6.8 g of 4-(diethylamino)-2-(2-ethylhexyloxy)benzaldehyde as a brown liquid in a yield of 86%.

¹H-NMR (CDCl₃): 10.17 (s, 1H), 7.69 (d, 1H), 6.24 (d, 1H), 6.00 (s, 1H), 3.90 (d, 2H), 3.41 (q, 4H), 1.74 (m, 1H), 1.64-1.29 (m, 8H), 1.20 (t, 6H), 0.94-0.86 (m, 6H)

### EXAMPLE 4

### Synthesis of 4-(diethylamino)-2-(cyclohexylmethoxy)benzaldehyde (3c of FIG. 1A)

16.2 g of 4-(diethylamino)-2-hydroxybenzaldehyde and 17.8 g of (bromomethyl)cyclohexane were put into 60 mL DMSO, 1.5 equivalents of sodium hydroxide was added thereto, followed by heating at 60_ for 8 hours. The resultant reactant was extracted with water and ethylacetate to remove an organic solvent, to obtain a solid. The obtained solid was washed with methanol and dried, to give 19.2 g of 4-(diethylamino)-2-(cyclohexylmethoxy)benzaldehyde in a yield of 86%.

¹H-NMR (CDCl₃): 10.18 (s, 1H), 7.69 (d, 1H), 6.24 (d, 1H), 5.97 (s, 1H), 3.79 (d, 2H), 3.39 (q, 4H), 1.87-1.71 (m, 6H), 1.27-1.10 (m, 11H)

### EXAMPLE 5

### Synthesis of 4-(diethylamino)-2-methylbenzaldehyde (3d of FIG. 1B)

10 mL POCl₃ was slowly added dropwise to 75 mL DMF (dimethyl formamide) at 0° C. After 30 minutes, 14.3 g of N,N-diethyl-m-toluidine was added thereto, followed by heating at 90° C. for 3 hours. The resultant reactant was cooled to room temperature and neutralized with sodium acetate and ice water. The resultant product was extracted with water and ethylacetate to remove an organic solvent, to give 13.5 g of 4-(diethylamino)-2-methylbenzaldehyde as a brown liquid in a yield of 81%.

¹H-NMR (CDCl₃) : 9.91 (s, 1H), 7.61 (d, 1H), 6.52 (d, 1H), 6.37 (s, 1H), 3.40 (q, 4H), 2.59 (s, 3H), 1.19 (t, 6H)

### EXAMPLE 6

### Synthesis of 1-hexylindoline-5-carbaldehyde (3e of FIG. 1C)

5.2 mL of POCl₃ was slowly added dropwise to 30 mL DMF at 0°. After 30 minutes, 10 g of hexylindoline was added thereto, followed by heating at 90° for 3 hours. The resultant reactant was cooled to room temperature and neutralized with sodium acetate and ice water, followed by subjecting to column chromatography using a mixed solvent of hexane and ethylacetate in a mixing ratio of 6:1 as an eluent, to give 5.2 g of 1-hexylindoline-5-carbaldehyde in a yield of 46%.

¹H-NMR (CDCl₃) : 9.60 (s, 1H), 7.50 (d, 1H), 7.49 (s, 1H), 6.31 (d, 1H), 3.56 (t, 2H), 3.16 (t, 2H), 3.00 (t, 2H), 1.57 (m, 2H), 1.30 (m, 6H), 0.87 (t, 3H)

### EXAMPLE 7

### Synthesis of 1-hexyl-1,2,3,4-tetrahydrocluinoline-6-carbaldehyde (3f of FIG. 1D)

3.6 mL of POCl₃ was slowly added dropwise to 20 mL DMF at 0°. After 30 minutes, 7.5 g of 1-hexyl-1,2,3,4-hydroquinoline was added thereto, followed by heating at 90° for 3 hours. The resultant reactant was cooled to room temperature and neutralized with sodium acetate and ice water. Then, the resultant product was extracted with water and ethylacetate to remove an organic solvent, to give 7.0 g of 1-hexyl-1,2,3,4-tetrahydroquinoline-6-carbaldehyde as a brown liquid in a yield of 82%.

¹H-NMR (CDCl₃): 9.62 (s, 1H), 7.52 (d, 1H), 7.50 (s, 1H), 6.53 (d, 1H), 3.36 (t, 2H), 3.29 (t, 2H), 2.75 (t, 2H), 1.92 (m, 2H), 1.59 (m, 2H), 1.33 (m, 6 H), 0.88 (t, 3H)

### EXAMPLE 8

### Synthesis of 1-(cyclohexylmethyl)-1 ,2,3,4-tetrahydroquinoline-6-carbaldehyde (3g of FIG. 1D)

6 mL of POCl₃ was slowly added dropwise to 45 mL DMF at 0°. After 30 minutes, 12 g of 1-cyclohexylmethyl-1,2,3,4-hydroquinoline was added thereto, followed by heating at 90° for 3 hours. The resultant reactant was cooled to room temperature and neutralized with sodium acetate and ice water. Then, the resultant product was extracted with water and ethylacetate to remove an organic solvent, to give 8.5 g of 1-cyclohexylmethyl-1,2,3,4-tetrahydroquinoline-6-carbaldehyde as a deep red liquid in a yield of 63%.

¹H-NMR (CDCl₃): 9.62 (s, 1H), 7.51 (d, 1H), 7.49 (s, 1H), 6.52 (d, 1H), 3.38 (t, 2H), 3.13 (d, 2H), 2.77 (t, 2H), 1.92 (m, 2H), 1.70 (m, 6H), 1.21 (m, 3H), 0.93 (m, 2H)

### EXAMPLE 9

### Synthesis of 1-(4-methylphenyl)-1,2,3,4-tetrahydroquinoline-6-carbaldehyde (3h of FIG. 1E)

2 mL of POCl₃ was slowly added dropwise to 15 mL DMF at 0°. After 30 minutes, 3.9 g of 1-(4-methylphenyl)-1,2,3,4-hydroquinoline was added thereto, followed by heating at 90° for 3 hours. The resultant reactant was cooled to room temperature and neutralized with sodium acetate and ice water, followed by subjecting to column chromatography using a mixed solvent of hexane and ethylacetate in a mixing ratio of 10:1 as an eluent, to give 2.2 g of 1-(4-methylphenyl)-1,2,3,4-tetrahydroquinoline-6-carbaldehyde in a yield of 50%.

¹H-NMR (CDCl₃) : 9.65 (s, 1H), 7.51 (s, 1H), 7.34 (d, 1H), 7.24 (d, 2H), 7.11 (d, 2H), 6.44 (d, 1H), 3.64 (t, 2H), 2.89 (t, 2H), 2.37 (s, 3H), 2.05 (m, 2H)

### EXAMPLE 10

### Synthesis of bis-DCMNEtOBu (1a of FIG. 1A)

0.94 g of (2,6-dimetyl-4H-pyran-4-ylidine)propanedinitrile (Formula 4) synthesized in Example 1, 3.0 g of 4-(diethylamino)-2-butoxybenzaldehyde (3a of FIG. 1A) synthesized in Example 2 and 0.6 mL pyperidine were put into 30 mL n-buthanol, followed by heating at 120° for 12 hours. After completing the reaction, the reactant was cooled to room temperature and excess methanol was added thereto to obtain a red solid. The obtained red solid was filtered and dried, followed by subjecting to column chromatography using methylenechloride as an eluent. Then, alcohol and methylenechloride were recrystallized to give 2.7 g of bis-DCMNEtOBu (1a of FIG. 1A) in a yield of 77%.

¹H-NMR (CDCl₃) :7.65 (d, 2H), 7.28 (d, 2H), 6.67 (d, 2H), 6.36 (s, 2H), 6.25 (d, 2H), 6.11 (s, 2H), 4.03 (t, 4H), 3.41 (q, 8H), 1.85 (m, 4H), 1.54 (m, 4H), 1.20 (t, 12H), 0.96 (t, 6H)

### EXAMPLE 11

### Synthesis of bis-DCMNEtOEH (1b of FIG. 1A)

0.81 g of (2,6-dimetyl-4H-pyran-4-ylidine)propanedinitrile (Formula 4) synthesized in Example 1, 3.2 g of 4-(diethylamino)-2-(2-ethylhexyloxy)benzaldehyde (3b of FIG. 1A) and 0.5 mL pyperidine were put into 30 mL n-buthanol, followed by heating at 120° for 12 hours. After completing the reaction, the reactant was cooled to room temperature and excess methanol was added thereto to obtain a red solid. The obtained red solid was filtered and dried, followed by subjecting to column chromatography using methylenechloride as an eluent and recrystallizing with alcohol and methylenechloride, to give 1.5 g of bis-DCMNEtOEH (1b of FIG. 1A) in a yield of 43%.

¹H-NMR (CDCl₃) :7.66 (d, 2H), 7.30 (d, 2H), 6.73 (d, 2H), 6.44 (s, 2H), 6.27 (d, 2H), 6.13 (s, 2H), 3.93 (m, 4H), 3.40 (q, 8H), 1.84 (m, 2H), 1.60-1.29 (m, 16H), 1.21 (t, 12H), 0.92 (t, 6H), 0.86 (t, 6H) EXAMPLE 12

### Synthesis of bis-DCMNEtOCy (1c of FIG. 1A)

1.0 g of (2,6-dimetyl-4H-pyran-4-ylidine)propanedinitrile (Formula 4) synthesized in Example 1, 3.7 g of 4-(diethylamino)-2-(cyclohexylmethoxy)benzaldehyde (3c of FIG. 1A) synthesized in Example 4, 0.6 mL pyperidine were put into 30 mL n-buthanol, followed by heating at 120° for 12 hours. After completing the reaction, the reactant was cooled to room temperature and excess methanol was added thereto to obtain a red solid. The obtained red solid was filtered and dried, followed by subjecting to column chromatography using methylenechloride as an eluent and recrystallizing with alcohol and methylenechloride, to give 3.6 g of bis-DCMNEtOCy (1c of FIG. 1A) in a yield of 87%.

¹H-NMR (CDCl₃) :7.70 (d, 2H), 7.31 (d, 2H), 6.70 (d, 2H), 6.43 (s, 2H), 6.27 (d, 2H), 6.09 (s, 2H), 3.82 (d, 4H), 3.40 (q, 8H), 1.90-1.76 (br, 6H), 1.72-1.68 (br, 4H), 1.64-1.61 (br, 2H), 1.29-1.06 (m, 22H)

### EXAMPLE 13

### Synthesis of bis-DCMNEtMe (1d of FIG. 1B)

2.0 g of (2,6-dimetyl-4H-pyran-4-ylidine)propanedinitrile (Formula 4) synthesized in Example 1, 4.9 g of 4-(diethylamino)-2-methylbenzaldehyde synthesized in Example 5 and 1.0 mL pyperidine were put into 40 mL n-buthanol, followed by heating at 120° for 12 hours. After completing the reaction, the reactant was cooled to room temperature and excess methanol was added thereto to obtain a red solid. The obtained red solid was filtered and dried, followed by subjecting to column chromatography using methylenechloride as an eluent and recrystallizing with alcohol and methylenechloride, to give 4.0 g of bis-DCMNEtMe (1d of FIG. 1B) in a yield of 66%.

¹H-NMR (CDCl₃) :7.73 (d, 2H), 7.49 (d, 2H), 6.54 (d, 2H), 6.43 (s, 2H), 6.42 (s, 2H), 6.38 (d, 2H), 3.40 (q, 8H), 2.41 (s, 6H), 1.20 (t, 12H)

### EXAMPLE 14

### Synthesis of bis-DCMIHex (1e of FIG. 1C)

0.95 g of (2,6-dimetyl-4H-pyran-4-ylidine)propanedinitrile (Formula 4) synthesized in Example 1, 2.8 g of 1-hexylindoline-5-carbaldehyde synthesized in Example 6 and 0.6 mL pyperidine were put into 30 mL n-buthanol, followed by heating at 120° for 12 hours. After completing the reaction, the reactant was cooled to room temperature and excess methanol was added thereto to obtain a red solid. The obtained red solid was filtered and dried, followed by subjecting to column chromatography using methylenechloride as an eluent and recrystallizing with alcohol and methylenechloride, to give 2.0 g of bis-DCMIHex (1e of FIG. 1B) in a yield of 61%.

¹H-NMR (CDCl₃) :7.32 (d, 2H), 7.24 (s, 2H), 7.20 (d, 2H), 6.39-6.33 (m, 6H), 3.52 (t, 4H), 3.14 (t, 4H), 3.01 (t, 4H), 1.57 (m, 4H), 1.39-1.31 (br, 12H), 0.90 (t, 6H)

### EXAMPLE 15

### Synthesis of bis-DCMQHex (1f of FIG. 1D)

1.4 g of (2,6-dimetyl-4H-pyran-4-ylidine)propanedinitrile (Formula 4) synthesized in Example 1, 4.4 g of 1-hexyl-1,2,3,4-tetrahydroquinoline-6-carbaldehyde synthesized in Example 7 (3f of FIG. 1D) 4.4 g and 0.8 mL pyperidine were put into 40 mL n-buthanol, followed by heating at 120° for 12 hours. After completing the reaction, the reactant was cooled to room temperature and excess methanol was added thereto to obtain a red solid. The obtained red solid was filtered and dried, followed by subjecting to column chromatography using methylenechloride as an eluent and recrystallizing with alcohol and methylenechloride, to give 3.0 g of bis-DCMQHex (1f of FIG. 1D) in a yield of 59%.

¹H-NMR (CDCl₃) :7.34 (d, 2H), 7.23 (d, 2H), 7.15 (s, 2H), 6.53 (d, 2H), 6.46 (s, 2H), 6.40 (d, 2H), 3.35 (t, 4H), 3.28 (t, 4H), 2.76 (t, 4H), 1.95 (m, 4H), 1.60 (m, 4H), 1.37-1.32 (br, 12H), 0.89 (t, 6H)

### EXAMPLE 16

### Synthesis of bis-DCMQCy (1q of FIG. 1D)

2.0 g of (2,6-dimetyl-4H-pyran-4-ylidine)propanedinitrile (Formula 4) synthesized in Example 1, 6.6 g of 1-cyclohexylmethyl-1,2,3,4-tetrahydroquinoline-6-carbaldehyde synthesized in Example 8 and 1.0 mL pyperidine were put into 40 mL n-buthanol, followed by heating at 120° for 12 hours. After completing the reaction, the reactant was cooled to room temperature and excess methanol was added thereto to obtain a red solid. The obtained red solid was filtered and dried, followed by subjecting to column chromatography using methylenechloride as an eluent and recrystallizing with alcohol and methylenechloride, to give 6.5 g of bis-DCMQCy (1g of FIG. 1D) in a yield of 86%.

¹H-NMR (CDCl₃) :7.34 (d, 2H), 7.22 (d, 2H), 7.15 (s, 2H), 6.52 (d, 2H), 6.45 (s, 2H), 6.40 (d, 2H), 3.38 (t, 4H), 3.11 (d, 4H), 2.77 (t, 4H), 1.96 (m, 4H), 1.89-1.70 (br, 12H), 1.20 (m, 6H), 0.98 (m, 4H)

### EXAMPLE 17

### Synthesis of bis-DCMQPhMe (1h of FIG. 1E)

0.62 g of (2,6-dimetyl-4H-pyran-4-ylidine)propanedinitrile (Formula 4) synthesized in Example 1, 2.0 g of 1-(4-methylphenyl)-1,2,3,4-tetrahydroquinoline-6-carbaldehyde synthesized in Example 9 and 0.4 mL pyperidine were put into 25 mL n-buthanol, followed by heating at 120° for 12 hours. After completing the reaction, the reactant was cooled to room temperature and excess methanol was added thereto to obtain a red solid. The obtained red solid was filtered and dried, followed by subjecting to column chromatography using methylenechloride as an eluent and recrystallizing with alcohol and methylenechloride, to give 1.1 g of bis-DCMQPhMe (1h of FIG. 1E) in a yield of 78%.

¹H-NMR (CDCl₃) :7.36 (d, 2H), 7.21 (d, 6H), 7.06 (m, 6H), 6.52 (s, 2H), 6.51 (d, 2H), 6.46 (d, 2H), 3.64 (t, 4H), 2.89 (t, 4H), 2.37 (s, 6H), 2.08 (m, 4H)

### EXAMPLE 18

### Synthesis of bis-DCJNBu (1i of FIG. 1F)

0.8 g of 4-(dicyanomethylene)-2-methyl-6-(julolidyn-4-yl-vinyl)-4H-pyrane (DCJ of FIG. 1F), 0.6 g of 4-(N,N′-dibutylamino)benzaldehyde and 0.4 mL pyperidine were put into 20 mL n-buthanol, followed by heating at 120_ for 12 hours. After completing the reaction, the reactant was cooled to room temperature and excess methanol was added thereto to obtain a red solid. The obtained red solid was filtered and dried, followed by subjecting to column chromatography using methylenechloride as an eluent and recrystallizing with alcohol and methylenechloride, to give 1.1 g of bis-DCJNBu (1i of FIG. 1F) in a yield of 86%.

¹H-NMR (CDCl₃): 7.41-7.28 (m, 4H), 6.99 (s, 2H), 6.92 (d, 2H), 6.45-6.37 (m, 4H), 3.32 (t, 4H), 3.25 (t, 4H), 2.75 (t, 4H), 1.96 (m, 4H), 1.61 (m, 4H), 1.39 (m, 4H), 0.96 (t, 6H)

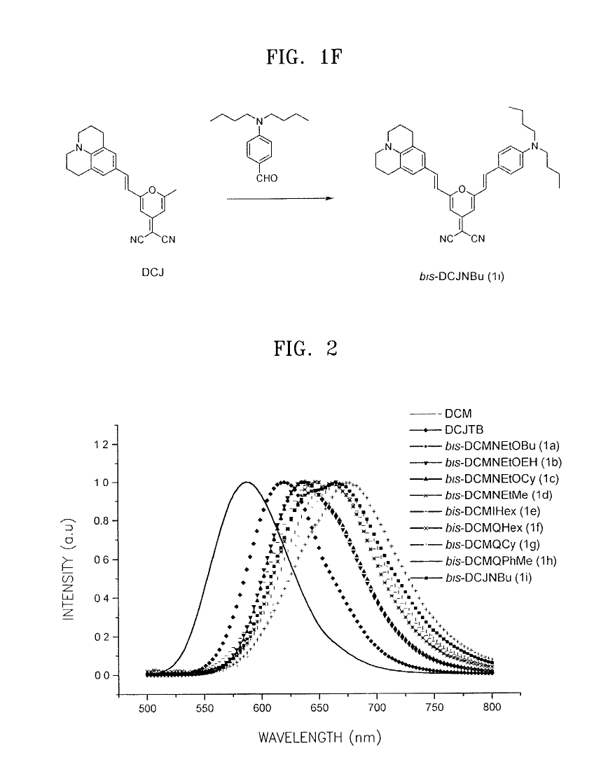

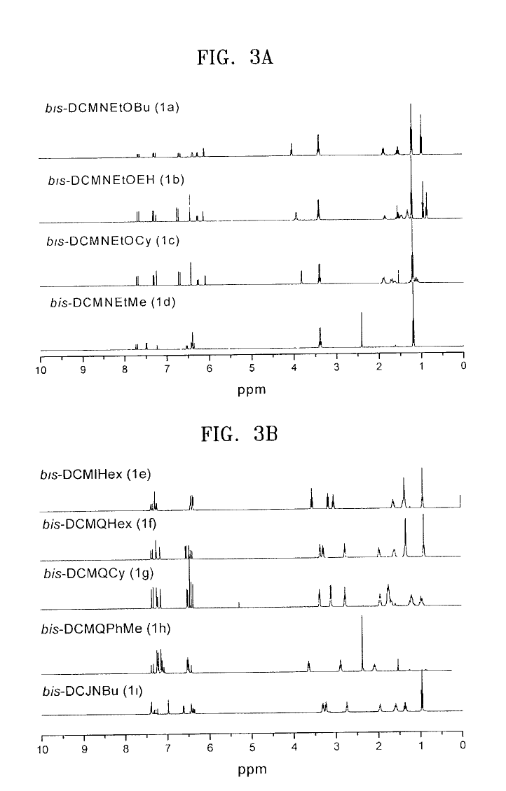

FIG. 2 is a ¹H-NMR spectrum illustrating red organic electroluminescent compounds synthesized in Examples 10 through 18 of the present invention. In FIG. 2, ¹H-NMR spectra of DCM as a conventional red light emitting material and DCJTB as another conventional red light emitting material substituted with t-butyl are also illustrated as comparative examples. FIGS. 3A and 3B are photoluminescent spectra in 1,2-dichloroethane illustrating red organic electroluminescent compounds synthesized in Examples 10 through 18 of the present invention. The red organic electroluminescent compounds according to the present invention emit light in a relatively long wavelength region being predominantly characteristic of red, compared to conventional red light emitting materials such as DCM, DCJTB and the like.

### EXAMPLE 19

### Fabrication of Organic Electroluminescent Device

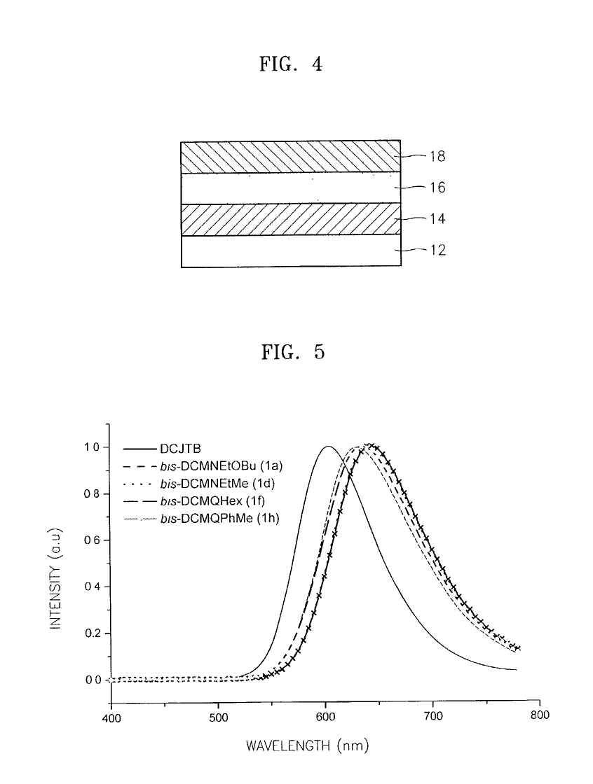

FIG. 4 is a cross-sectional view for explaining a method for fabricating an organic electroluminescent device according to the present invention. Referring to FIG. 4, an anode 14 made of ITO (indium tin oxide) was formed on a glass substrate 12. Then, a chloroform solution dissolved by doping each 2 wt % of bis-DCMNEtOBu (1a of FIG. 1A) synthesized in Example 10, bis-DCMNEtMe (1d of FIG. 1B) synthesized in Example 13, bis-DCMQHex (1f of FIG. 1D) synthesized in Example 15, and bis-DCMQPhMe (1e of FIG. 1E) synthesized in Example 17 into poly(N-vinylcarbazole) (PVK), was spin-coated on the anode 14 to a thickness of 100 nm to form an emitting layer 16. Thereafter, an Al film as a cathode layer was deposited on the emitting layer 16 to a thickness of 100 nm to form a cathode 18. During deposition for forming the cathode 18, the degree of vacuum was maintained at 1×10⁻⁵ Torr or less.

FIG. 5 is an electroluminescence (EL) spectrum of various organic electroluminescent devices according to the present invention. In FIG. 5, EL spectral distribution of an EL device using a conventional red light emitting material DCJTB is also illustrated as a comparative example. Table 1 shows color coordinates (1931 CIE) corresponding to the result shown in FIG. 5. Like in photoluminescence, the organic EL device according to the present invention emits red light with higher purity compared to the conventional organic EL device using DCJTB.

TABLE 1

| | λ_{PL} (nm)^{a} | λ_{EL} (nm)^{b} | 1931 CIE (x,y)^{c} |
|---|---|---|---|
| DCJTB | 621 | 604 | (0.59, 0.40) |
| bis-DCMNEtOBu (1a) | 637 | 630 | (0.64, 0.35) |
| bis-DCMNEtNMe (1d) | 649 | 642 | (0.65, 0.34) |
| bis-DCMQHex (1f) | 667 | 644 | (0.66, 0.34) |
| bis-DCMQPhMe (1h) | 646 | 634 | (0.64, 0.36) |

In Table 1, a represents measurement in a 1,2-dichloroethane solution, b represents emission spectrum of the organic EL device fabricated in Example 19, and c represents color coordinates given by the EL spectrum, where x+y+z=1, x, y and z are red, green and blue proportions.

### EXAMPLE 20

### Fabrication of organic electroluminescent device

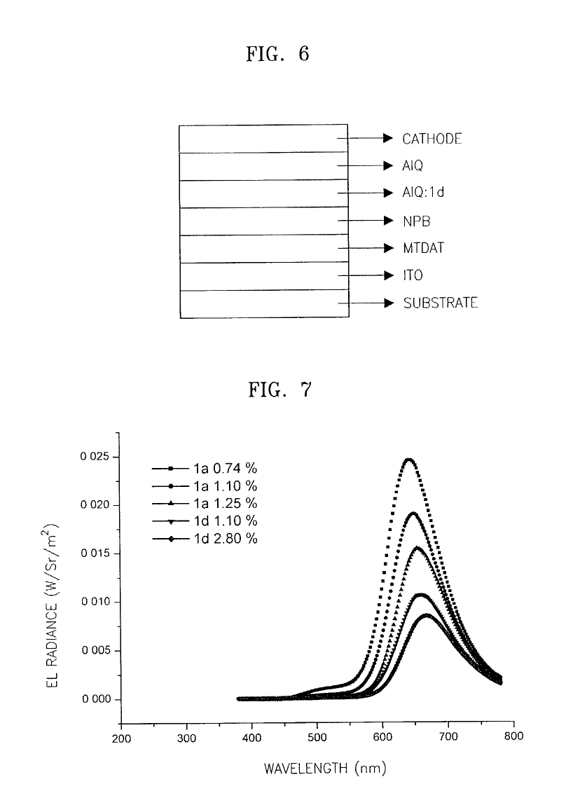

As shown in FIG. 6, a device having a laminated structure of ITO/MTDATA(200 Å)/NPB(400 Å)/Alq3-red emitting material (300 Å)/Alq3(300 Å)/LiF(10 Å)/Al(1000 Å) was vacuum deposited under a pressure of 10⁻⁶ torr or less to fabricate a red organic electroluminescent device.

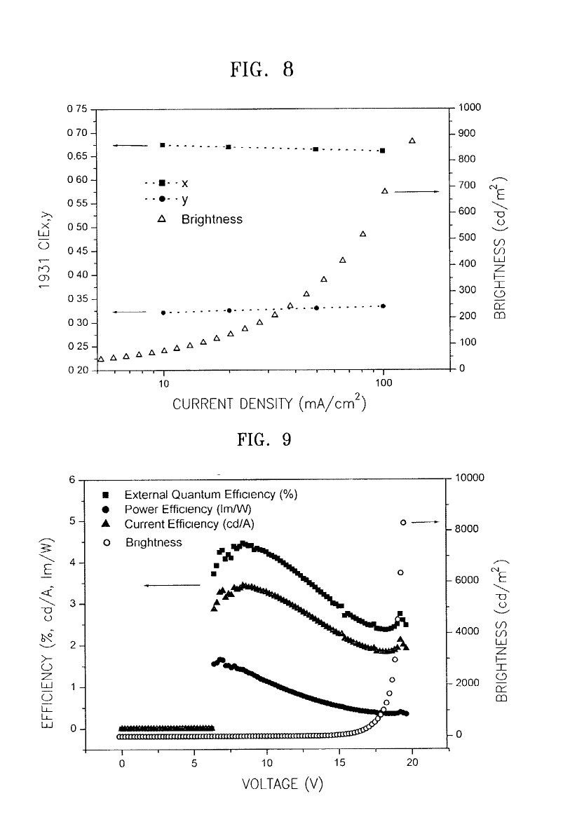

FIG. 7 is an EL spectrum of the red organic electroluminescent device shown in FIG. 6, in which the spectral distribution is measured at a current density of 20 mA/cm². Referring to FIG. 7, as the density of bis-DCMNEtOBu (1a) doped into Alq3 was gradually increased to 0.74 wt %, 1.0 wt % and 1.15 wt %, the maximum luminescence wavelength shifted to longer wavelength regions, that is, to 643 nm, 649 nm and 654 nm. Also, the (x,y) color coordinates in the 1931 Commission Internationale de I'Eclairage (CIE) chromaticity diagram were (0.63, 0.36), (0.65, 0.34), (0.67, 0.33), which was very near the pure red color coordinates (0.67, 0.33) requested by the National Television System Committee (NTSC). While the EL device doped with bis-DCMNEtMe (1d) at doping concentrations of 1.10 wt % and 2.80 wt % had the maximum luminescence efficiency and good color coordinates (0.66, 0.33) at relatively longer wavelengths of 659 nm and 668 nm, it had very low current efficiencies, that is, 0.49 cd/A and 0.29 cd/A. Evaluation results of performance characteristics of the EL devices evaluated in FIG. 7 are listed in Table 2.

TABLE 2

| | | | | | | | Maximum | |
|---|---|---|---|---|---|---|---|---|
| | | | | | | | brightness | |
| | Doping | Bright- | 1931 | | | | (Voltage (V), | Maximum |
| | concen- | ness, | CIE | | _em, | Radiance | Current | efficiency |
| Red | tration | (cd/m²) | (x,y) | cd/A | nm | (W/Sr/m²) | density (mA/c | (%, cd/A, |
| dopant | % | [a] | [a] | [a] | [a] | [a] | m²)) cd/m² | Im/W) |
| 1a | 0.74 | 397 | (0.63, 0.36) | 1.99 | 643 | 2.68 | 8288 (19.6, 434) | 4.46, 3.43, 1.64 |
| 1a | 1.1 | 236 | (0.65, 0.34) | 1.18 | 649 | 1.99 | 5067 (21.4, 394) | 4.42, 2.91, 1.69 |
| 1a | 1.25 | 137 | (0.67, 0.33) | 0.69 | 654 | 1.56 | 2501 (21.2, 423) | 2.95, 1.29, 0.99 |
| 1d | 1.1 | 97 | (0.66, 0.33) | 0.49 | 659 | 1.14 | 3538 (15.2, 535) | 2.10, 1.00, 0.84 |
| 1d | 2.8 | 57 | (0.67, 0.32) | 0.29 | 668 | 0.89 | 1439 (18.2, 641) | 1.81, 0.59, 0.58 |

In Table 2, “a” represents measurement at 20 mA/cm².

FIG. 8 is a graphical representation of current dependent color stability of the organic EL device doped with 1.25 wt % of bis-DCMEtOBu exhibiting substantially the same color purity level as that of NTSC red emission. The EL device had very high brightness of several hundreds cd/m² while maintaining stable color purity at practically applicable current density areas of several mA/cm² to several tens mA/cm².

FIG. 9 is a graphical representation of voltage efficiencies of the organic EL device doped with 0.74 wt % of bis-DCMNEtOBu. Referring to FIG. 9, the external quantum efficiency was approximately 4.46% around 7 V, the current efficiency was approximately 3.43 cd/A, and power efficiency was approximately 1.64 Im/W. The maximum brightness was greater than 8000 cd/m², which is a very high level, suggesting that this material is quite good as an organic EL material.

Although only an emitting layer having a single layered structure has been described with reference to FIG. 4 and FIG. 6 illustrating a method of fabricating an organic EL device, the present invention is not limited thereto. As is well known to one skilled in the art, the organic EL device according to the present invention may comprise multi-layers consisting of a hole transporting layer, an emitting layer and an electron transport layer, as described with reference to FIG. 4. White emission can also be realized by mixing the red organic electroluminescent compound according to the present invention with other color compounds in forming the emitting layer.

As described above, according to the present invention, in order to synthesize an organic electroluminescent compound capable of realizing red emission with higher purity, a DCM derivative further having an electron donor in an aryl ring having an amine group as an electron donor, is synthesized, thereby providing pure red light emitting properties and good luminescence efficiency. Also, in the organic EL device according to the present invention, a uniformly thick film can be formed by vacuum deposition. The organic EL device according to the present invention includes an emitting layer having red organic electroluminescent compounds capable of providing pure red light emitting properties and good luminescence efficiency. Accordingly, the organic EL device also has good color coordinates and provides pure red light emitting properties and good luminescence efficiency, compared to the case of using a conventional red light emitting material, thereby being industrially advantageously used.

While the present invention has been particularly shown and described with reference to preferred embodiments thereof, it will be understood by those of ordinary skill in the art that various changes in form and details may be made therein without departing from the spirit and scope of the present invention as defined by the following claims.

We claim:

1. A red organic electroluminescent compound having the formula: 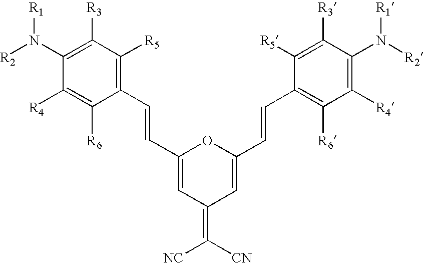
wherein R₁, R₁′, R₂ and R₂′ are independently hydrogen atom, or C_{1—}C₃₀ alkyl, aryl or hetero ring; R₃, R₃′, R₄ and R₄′ are independently hydrogen atom, C_{1—}C₁₀ alkyl or alkoxy; one or more pairs selected from the group consisting of R₁ and R₃, R₁′ and R₃′, R₂ and R₄, and R₂′ and R₄′ can be connected in forms of —R₁-R₃—, —R₁′-R₃′—, —R₂-R₄—, and —R₂′-R₄′—; R₅, R₅′, R₆ and R₆′ are independently hydrogen atom, or C_{1—}C₃₀ alkyl, alkoxy or aryl; at least one of R₃, R₃′, R₄, R₄′, R₅, R₅′, R₆ and R₆′ is not hydrogen atom.

2. The red organic electroluminescent compound according to claim 1, wherein the one or more pairs selected from the group consisting of R₁ and R₃, R₁′ and R₃′, R₂ and R₄, and R₂′ and R₄′ can be connected in forms of —R₁-R₃—, —R₁′-R₃′—, —R₂-R₄—, and —R₂′-R₄′—, thereby forming the structure of —CR₇R₈—(CR₉R₁₀)ₘ—CR₁₁R₁₂—, and wherein R₇, R₈, R₉, R₁₀, R₁₁ and R₁₂ are independently hydrogen atom or C_{1—}C₄ alkyl, and m is an integer between 0 and 2.

3. The red organic electroluminescent compound according to claim 1, wherein R₁, R₁′, R₂ and R₂′ are independently methyl, ethyl, propyl, butyl, pentyl, hexyl, aryl, dialkylfluoryl or heteroaryl.

4. The red organic electroluminescent compound according to claim 1, wherein R₃, R₃′, R₄ and R₄′ are independently hydrogen atom, methyl, ethyl, propyl, butyl or alkoxy.

5. The red organic electroluminescent compound according to claim 1, wherein R₅, R₅′, R₆ and R₆′ are independently hydrogen atom, methyl, ethyl, propyl, butyl, methoxy, ethoxy, butoxy, cyclohexylmethoxy or ethylhexyloxy.

6. The red organic electroluminescent compound according to claim 1 having the formula:

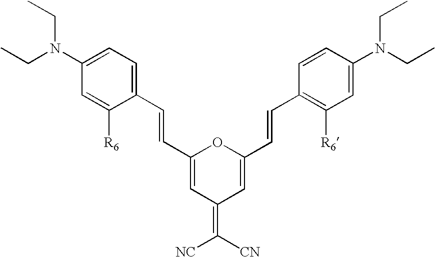

7. The red organic electroluminescent compound according to claim 6, wherein R₆ and R₆′ are independently methyl, ethyl, methoxy, ethyoxy, propyloxy, butoxy, cyclohexylmethyloxy or ethylhexyloxy.

8. The red organic electroluminescent compound according to claim 1 having the formula: 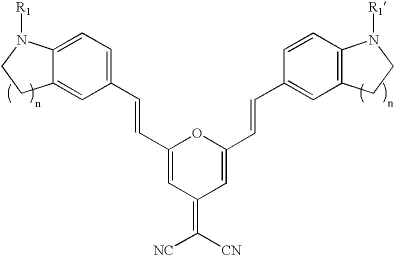
wherein n is an integer between 0 and 3.

9. The red organic electroluminescent compound according to claim 8, wherein R₁ and R₁′ are independently methyl, ethyl, cyclohexyl, hexyl, methylphenyl or dialkylfluoryl, and n is 1 or 2.

10. A method for synthesizing the red organic electroluminescent compound according to claim 1, the method comprising:
(a) preparing a first compound having the formula: 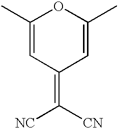
(b) reacting the first compound with a second compound having the formula

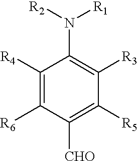

11. The method according to claim 10, wherein in step (b), a third compound having the following formula is recovered as a product of the reaction between the first compound and the second compound: 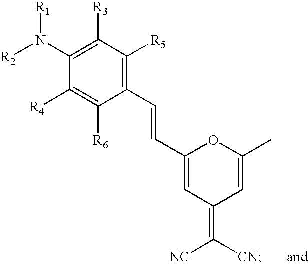
the method further comprising (c) reacting the third compound with a fourth compound having the formula:

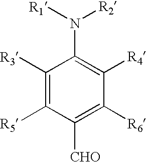

12. An organic electroluminescent device comprising:
an anode;
a cathode; and
an emitting layer interposed between the anode and the cathode and having the red organic electroluminescent compound according to claim 1.

13. An organic electroluminescent device comprising:
an anode;
a cathode; and
an emitting layer interposed between the anode and the cathode and having the red organic electroluminescent compound according to claim 2.

14. An organic electroluminescent device comprising:
an anode;
a cathode; and
an emitting layer interposed between the anode and the cathode and having the red organic electroluminescent compound according to claim 3.

15. An organic electroluminescent device comprising:
an anode;
a cathode; and
an emitting layer interposed between the anode and the cathode and having the red organic electroluminescent compound according to claim 4.

16. An organic electroluminescent device comprising:
an anode;
a cathode; and
an emitting layer interposed between the anode and the cathode and having the red organic electroluminescent compound according to claim 5.

17. An organic electroluminescent device comprising:
an anode;
a cathode; and
an emitting layer interposed between the anode and the cathode and having the red organic electroluminescent compound according to claim 6.

18. An organic electroluminescent device comprising:
an anode;
a cathode; and
an emitting layer interposed between the anode and the cathode and having the red organic electroluminescent compound according to claim 7.

19. An organic electroluminescent device comprising:
an anode;
a cathode; and
an emitting layer interposed between the anode and the cathode and having the red organic electroluminescent compound according to claim 8.

20. An organic electroluminescent device comprising:
an anode;
a cathode; and
an emitting layer interposed between the anode and the cathode and having the red organic electroluminescent compound according to claim 9.
